(19) United States
(12) Patent Application Publication
(10) Pub. No.: US 2011/0184352 A1
(43) Pub. Date: Jul. 28, 2011

(54) Universal traveler's syringe for vaginal feminine hygiene

(76) Inventor: Liberty Bernardino Uy, Daly City, CA (US)

(21) Appl. No.: 12/800,074

(22) Filed: May 7, 2010

Related U.S. Application Data
(60) Provisional application No. 61/336,953, filed on Jan. 28, 2010.

(51) Int. Cl. A61M 3/02

(52) U.S. Cl. 604/257

(57) ABSTRACT

A method and apparatus for the cleansing of the vagina using an L-shape disposable syringe with a cap or closure assembly that communicates to any standard bottled water that is available in the market.

### CROSS REFERENCE TO RELATED APPLICATIONS

This application claims the benefit of the provisional patent application Ser. No. 61/336,953, filed 2010 Jan. 28 by Liberty B. Uy

### BACKGROUND OF THE PRESENT INVENTION

Vagina irrigation devices have gained wide use in cleansing and treating the female organs. One such system previously known reusable douche have been particularly inconvenient for the user when away from home due to the bulkiness of the equipment and the maintenance of cleaning the device due to the frequent lack of facilities necessary to perform these operations, especially when traveling.

Disposable douches are also inconvenient to use due to its bulkiness and the need to assemble many parts during use.

In addition to the disadvantages of previous inventions, most disposable pre-mix douches are not allowed on airplanes because of the evolving security threats to aviation.

### SUMMARY

This invention may be summarized at least in part with reference to its objective. It is the primary objective of the present invention to provide the most convenient, effective, sanitary, safe to carry, and handy way to douche conveniently without the bulkiness of the kit. All you need to carry is the present invention! When you are ready to use it, just connect it to any standard bottled water or any bottle that has the same dimension.

The present invention can be manufactured as a soft flexible material to allow the user to adjust the syringe to the most comfortable position to avoid discomfort or injury. Therefore, it doesn't limit the manufacturing material to plastic.

In addition to the present invention it has a unique L-shape profile designed so that the bottle will be positioned upwardly for ease of grip and a more effective usability.

Also, this invention can simply be produced, providing inexpensive syringes for healthy vaginal hygiene.

### DRAWINGS—FIGURES

FIG. 1 Is the Perspective View of a syringe for vaginal irrigation in accordance with the preferred embodiment of the present invention. It shows combined Syringe for fitting on bottled water, showing my new design. (The bottled water illustrated as broken lines is not part of the claimed design.)

FIG. 2 The Side View of the syringe of FIG. 1

FIG. 3 The Top View of the syringe of FIG. 1

FIG. 4 The 3D View of the syringe of FIG. 1

FIG. 5 The Exploded View of the syringe of FIG. 1

### DETAILED DESCRIPTION OF THE INVENTION

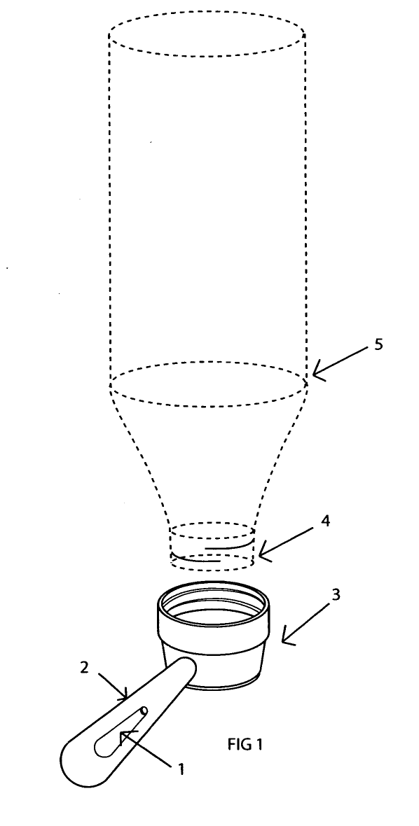

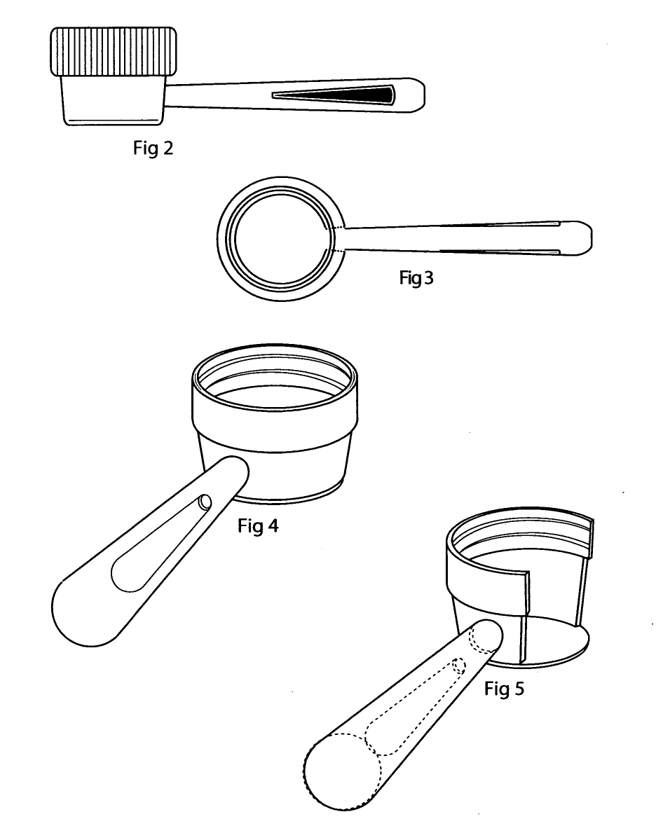

Referring now to the drawings and particularly FIGS. 1, the preferred embodiment of the invention, generally designated FIG. 1-3 is illustrated that the cap 3 or closure assembly communicates to any standard bottled water 5. The syringe 2 comprises a hollow vaginal insert. When syringe 2 is completely assembled to the neck 4 of the standard bottled water 5, the position must be completely overturned before the syringe 2 is inserted into the vagina. Such a completely overturned position of a standard water bottle 5 allows the water to naturally flow down to the cap 3, then to the syringe 2, then to the ejection hole 1 due to gravity. In addition, when bottled water 5 is compressed by hand, the water due to pressure flows from standard bottled water 5, to the cap 3, to the syringe 2 then flows both to the ejection hole 1. Suitable pressure may then be applied to provide the desired flow rate of water until the standard bottled water 5 is empty. The syringe 2 may then be removed and disposed.

As describe above, the vaginal syringe is actually only one piece which has a cap that communicates to any standard bottled water. The syringe has an L-shaped profile when the syringe is completely assembled to standard water bottle water. Therefore, position must be completely overturned before the syringe is inserted into the vagina. The water naturally flow down to the cap, then to the syringe, then to the ejection holes. Also, When compressed by hand, the water due to pressure flow from standard bottled water, to the cap, to the syringe, then flows both to the ejection holes.

While a full and complete description of the invention has been set forth in accordance with the dictates of the Patent Statutes, it is to be understood that the invention is not intended to be limited to the specific form herein shown.

Accordingly, modification of the invention may be shown without departing from the spirit hereof or the scope of the accompanying claims.

I claim:

1. A syringe for vaginal irrigation, compromising:
A disposable L-shape profile syringe that has a cap that communicates on the opening of all standard bottled water example given like Evian, Crystal Geyser, Calistoga or any water bottle that is available in the market or any bottle that fits our variety of dimension.

2. I claim the ornamental design for a combined syringe and bottle cap for fitting on water bottles, as shown and described

3. The claim 1 can be manufactured in plastic (polyethylene) however; it is not limited to plastic and can be manufactured in rubber or in any other materials.

4. The claim 1 is preferably manufactured in white color; however, it doesn't limit it to be manufactured in any color

5. The irrigation nozzle in FIG. 1-1 is provided with two ejection holes on the side however, it can also be manufactured with plurality of apertures.
